(19) United States
(12) Patent Application Publication
(10) Pub. No.: US 2022/0211538 A1
(43) Pub. Date: Jul. 7, 2022

(54) MALE EXTERNAL CATHETER

(71) Applicant: Venkata Buddharaju, Park Ridge, IL (US)

(72) Inventor: Venkata Buddharaju, Park Ridge, IL (US)

(21) Appl. No.: 17/702,490

(22) Filed: Mar. 23, 2022

Related U.S. Application Data
(63) Continuation-in-part of application No. 17/317,457, filed on May 11, 2021.
Application No. 17/317,457 is a continuation-in-part of application No. 17/132,842, filed on Dec. 23, 2020, now Pat. No. 11,337,848.

(51) Int. Cl. A61F 5/453; A61F 5/44; A61F 5/443

(52) U.S. Cl. CPC A61F 5/453; A61F 5/443; A61F 5/4408

(57) ABSTRACT

An attachment device for securing a medical device to a user includes a supportive strap portion and an adhesive body. The supportive strap portion includes a first strap end and a second strap end opposite to the first strap end. The first strap end includes a first fastener providing a first fastening point, and the second strap end includes a second fastener providing a second fastening point. The supportive strap portion is elastic and has an unstretched length between the first and second fastening points of the first and second strap ends. The adhesive body is secured to the second strap end using the second fastener. The first strap end is connected to the medical device using the first fastener.

### CROSS REFERENCE

The present application comprises a continuation-in-part of U.S. application Ser. No. 17/317,457 filed May 11, 2021, which is a continuation-in-part of U.S. application Ser. No. 17/132,842 filed Dec. 23, 2020, the entire contents of which are incorporated herein by reference.

### BACKGROUND

The present disclosure relates to male external catheters. More specifically, the present disclosure is directed to a male external catheter including a flexible flange on a tubular body having varying wall thicknesses, wherein the flexible flange is deformed to provide a secure fit during use.

Condom catheters are alternatives to indwelling catheters that are less invasive to the patient. A conventional condom catheter is a sheath worn about the penis attached to tubing that directs urine to a drainage bag. The sheath is typically made of silicone and/or latex and includes a uniform wall thickness throughout the height.

An adhesive is typically provided on the interior of the opening of the sheath to adhere the condom catheter to the user's penis. In some catheters, a loop and pile (Velcro™) tape releaseable fastener or other fastener may wrap around the body of the catheter near the base thereof to secure the catheter about the penis.

Users of conventional condom catheters often experience pain or discomfort during use. The latex material may irritate the user's skin, and users often cannot anticipate having a reaction until the catheter is applied to the penis. Further, adhesives and other elements used to secure the catheter in place on the penis often get caught on the user's sensitive skin and hair within the region, leading to more acute pain. The adhesive material often times attaches too hard and tight around the penile skin, causing swelling, skin damage, and discomfort during use and/or removal. When left in place for too long, localized skin removal and/or wound formation may result, causing the patient to require the insertion of an indwelling catheter into the urethra and bladder for urinary drainage. Indwelling catheters increase the risk for urinary tract infections.

Another significant problem faced by condom catheters is leakage as well as difficulty retaining the condom catheter in place. Prior art systems including mechanisms to secure the condom catheter in place, such as complex belts with straps and/or adhesives. Belt systems tend to be cumbersome and uncomfortable, and do not ensure that leakage is avoided. Similarly adhesives may include gaps formed during application through which urine escapes. When urine and sweat interact with the adhesives, which is especially common in the pubic area having hair, the adhesive can become dislodged, resulting in leakage and needing to be replaced frequently, causing further pain and discomfort as well as increased health care costs.

Current condom catheters face the following challenges: (1) keeping the device in place longer; (2) using an adhesive near urine and sweat, which compromises the stability of the catheter; (3) the constrictive effect of adhesive on expanding penile skin with risk to skin injury; and (4) condom catheters that are currently being used have to be replaced each time the catheter slips off, leading to increased healthcare costs.

Accordingly, there is a need for a male external catheter that overcomes these challenges and that minimizes leakage without utilizing adhesives or other additional components on the body of the condom catheter, as described herein.

### SUMMARY

To meet the needs noted above and others, the present disclosure provides a male external catheter that conforms or adapts to the size and fit around the user's penis as urine flows through, thereby maintaining its positioning without the need for adhesives or other securing mechanisms on penile skin or near hairy pubic area on in close contact with urine while reducing the likelihood of leakage. The male external catheter includes a flexible flange surrounding an opening into the body of the catheter. First and second attachment devices utilizing supportive strap portions attach to opposite sides of the flexible flange and are pulled taught, upwardly and outwardly away from the catheter, and include adhesives on outer ends thereof that are secured to the user's body. The presently claimed male external catheter provides an improved hold on the penis by including adhesives that adhere to the patient's body at a distance from the user's penis, which exerts an even force on both sides of the flange of the condom catheter to center the catheter on the user's penis for improved stability, functionality, and patient comfort. The male external catheter described herein is reusable, reducing waste as well as healthcare costs.

The body of the male external catheter has a tubular shape with a first end having a first opening through which the penis extends during use. A second end of the body has a second opening that connects to tubing of a drainage bag. A flexible flange at the first end of the tubular body surrounds the first opening and includes a first side portion and a second side portion on opposing sides of the flange. Each attachment device includes a supportive strap portion that attaches to the body at a first end and an adhesive at a second end. Specifically, the first end of the first supportive strap portion connects to the first side portion of the flange at a first inner end, and the second end of the second supportive strap portion connects to the second side portion of the flange at a second inner end.

Adhesives attached to the second ends of the first and second supportive strap portions, respectively, are secured to the user's body at a distance from the body of the male external catheter. During use, the second ends of the supportive strap portions are pulled away from the tubular body of the catheter, for example, at 45 degree angles, and attached to the user's body near the lower abdomen or lower anterior hip area spaced from the groin, preferably in areas that are relatively free from hair. By pulling upwards and away from the tubular body, equal forces are applied to the sides of the flange, centering the tubular body on the user and securely holding the tubular body in place.

The first and second ends of the supportive strap portions include fasteners such as snap buttons that allow for easy attachment and detachment of the supportive strap portion to the adhesive and the body of the male external catheter. In some embodiments, the snap button includes a male component spaced from a plurality of female components so that the user can modify the length of the supportive strap portion and/or location of the adhesive on the body.

In some example use cases, the user is a patient with limited mobility that requires a healthcare worker to manipulate the patient's body. The use of the first and second supportive strap portions with adhesives attached to the user at a distance from the tubular member secures the tubular body in place as the patient is shifted and/or rotated.

In one embodiment, the first and second side portions include first and second strap openings, respectively. The first and second supportive strap portions extend through first and second strap openings, respectively, of the first and second side portions, respectively, of the flange. In one embodiment, the first and second supportive strap portions are part of a single strap, and the strap includes a third portion between the first and second supportive strap portions. The third supportive strap portion extends below the flange along a back side of the body during use.

The flange comprises a flexible material that deforms under pressure. In some embodiments, the flange is a silicone material, although other suitable materials may be used as desired. The flange may be free from latex and/or adhesive.

A further benefit of the present design over the use of conventional catheters that utilize adhesives is the ability to temporarily allow airflow in through the flange of the catheter to allow the column of urine to drain from the tip of the catheter into the drainage bag. Without allowing air to enter the catheter, the column of urine remains in place between the catheter tip and the drainage bag. In the present design, the user can gently lift the side portions of the flange to allow airflow around the penis while the male external catheter is attached to the user, enabling the column of urine to drain into the drainage bag. The user can then allow the catheter to return to the original position. In conventional adhesive catheters, the column of urine stays in contact with the skin and adhesive, risking the loss of adhesive by constant urine contact and causing softening of the skin and related skin loss. The adhesive catheter must be replaced altogether once the adhesive surface is separated from the skin, whereas the presently described male external catheter can be easily removed and washed for reuse.

In some embodiments, the body varies in cross-sectional area along a height thereof. The body includes a base portion, a central portion, and a tip portion, with the base portion being adjacent to the base and the tip portion configured to connect to tubing of a drainage bag. Each of the base portion, the central portion, and the tip portion include a base wall thickness, a central wall thickness, and a tip wall thickness, respectively. The central wall thickness is less than each of the base wall thickness and the tip wall thickness. This difference in wall thickness enables the central portion of the sheath to collapse around the user's penis while urine flows through the sheath and tip into the drainage bag.

The base portion of the body may be formed integrally with the flange. In some embodiments, the base portion has a base height h_{b} of approximately 0.830 in, although the base height h_{b} may vary as desired or required during manufacturing. The base wall thickness t_{b} is preferably about 0.060 in at the greatest point, although the base wall thickness t_{b} may vary as desired or as required during manufacturing. In some embodiments, the base wall thickness t_{b} tapers from a predominant base wall thickness t_{b-p} to the central wall thickness t_{c} near the juncture of the base portion 102a and the central portion 102b. In one embodiment, the predominant base wall thickness t_{b-p} is about 0.060 in. In one embodiment, the base wall thickness tapers from a predominant base wall thickness to the central wall thickness near the juncture of the base portion and the central portion.

The central portion of the body includes a planar section adjacent to the base portion and a bellowed section adjacent to the tip portion. The bellowed section may include one or more horizontal bellows transverse to the height H of the body. In some embodiments, the planar section may include optional vertical ribbing leading up to and connected to the horizontal bellows. The vertical ribs may be evenly positioned along the perimeter of the body at or near the juncture of the planar section and the bellowed section. The central portion has a central height h_{c} of about 1.514 in, although the central height h_{c} may vary as desired or required during manufacturing. The central wall thickness t_{c} is preferably about 0.030 in, although the central wall thickness t_{c} may vary as desired or as required during manufacturing.

The tip portion of the body has a tip height hₜ of about 1.315 in, although the tip height hₜ may vary as desired or required during manufacturing. The tip wall thickness tₜ is preferably about 0.060 in although the tip wall thickness tₜ may vary as desired or required during manufacturing. In some embodiments, the tip portion includes horizontal ribbing along the perimeter of the cylindrical body.

In the embodiments described herein, the condom catheter can be reused for up to at least five days if washed daily. The supportive strap portions may be elastic and/or latex-free. Silicone adhesive bodies are soft on the skin, easy to apply and reapply, particularly on non-hairy portions of the body such as the hip area, and are more comfortable as compared to adhesives used in conventional condom catheters attached to the penile skin, which are more painful to remove due to sensitive skin and entanglement with pubic hair. The adhesive body attached to the second strap end can be repositioned and maintain its adhesive integrity up to at least five days. Depending on the user needs, both the supportive strap portions and/or the adhesive bodies can be replaced as needed using the snap buttons during the five days reuse of the condom catheter.

In light of the disclosure set forth herein, and without limiting the disclosure in any way, in a first aspect, which may be combined with any other aspect or portion thereof described herein, an attachment device for securing a medical device to a user includes a supportive strap portion and an adhesive. The supportive strap portion includes a first strap end and a second strap end opposite to the first strap end. The first strap end includes a first fastener providing a first fastening point, and the second strap end includes a second fastener providing a second fastening point. The supportive strap portion is elastic and has an unstretched length between the first and second fastening points of the first and second strap ends. The adhesive body is secured to the second strap end using the second fastener, and the first strap end is connected to the medical device using the first fastener.

In a second aspect, which may be combined with any other aspect or portion thereof described herein unless otherwise specified, the first fastener comprises a first snap button that includes a male component and a female component spaced apart on the supportive strap portion adjacent to the first strap end, and/or the second fastener comprises a second snap button that includes a male component and a female component spaced apart on the supportive strap portion adjacent to the second strap end. In some embodiments, each of the first and second snap buttons includes a plurality of female components positioned along the supportive strap portion.

In a third aspect, which may be combined with any other aspect or portion thereof described herein unless otherwise specified, the first strap end of the supportive strap portion is positioned within an opening of the medical device such that the male and female components are positioned on opposite sides of the opening of the medical device and secured together to form the first fastening point.

In a fourth aspect, which may be combined with any other aspect or portion thereof described herein unless otherwise specified, the adhesive body includes an opening. The second strap end of the supportive strap portion may be positioned within the opening of the adhesive body such that the male and female components are positioned on opposite sides of the opening of the adhesive body and secured together to form the second fastening point.

In a fifth aspect, which may be combined with any other aspect or portion thereof described herein unless otherwise specified, the attachment device further comprises a further supportive strap portion and a further adhesive body. The further supportive strap portion includes a further first strap end and a further second strap end opposite to the further first strap end. The further first strap end includes a first fastener providing a first fastening point, and the further second strap end includes a second fastener providing a second fastening point. The further supportive strap portion is elastic and has an unstretched length between the first and second fastening points of the further first and second strap ends. The further adhesive body is secured to the further second strap end using the second fastener.

In a sixth aspect, which may be combined with any other aspect or portion thereof described herein unless otherwise specified, the medical device comprises one of a male external catheter, a tracheostomy tube, and an endotracheal tube.

In a seventh aspect, which may be combined with any other aspect or portion thereof described herein unless otherwise specified, a method for securing a medical device to a user's body is provided. The method comprises the steps of providing an attachment device comprising a supportive strap portion and an adhesive. The supportive strap portion includes a first strap end and a second strap end opposite to the first strap end. The first strap end includes a first fastener providing a first fastening point, and the second strap end includes a second fastener providing a second fastening point. The supportive strap portion is elastic and has an unstretched length between the first and second fastening points of the first and second strap ends. The adhesive body is secured to the second strap end using the second fastener. The method further comprises the steps of connecting the first strap end of the supportive strap portion to the medical device using the first fastener; positioning the medical device on the user's body; and securing the adhesive body to the user's body. A distance between the adhesive body secured to the user's body and the medical device positioned on the user's body causes a distance between the first and second fastening points of the supportive strap portion to be greater than the unstretched length of the supportive strap portion, creating tension along the supportive strap portion that secures the medical device to the user's body.

In an eighth aspect, which may be combined with any other aspect or portion thereof described herein unless otherwise specified, the method further comprises positioning the medical device on the user's body before securing the adhesive body to the user's body; and pulling the adhesive body and the second strap end away from the medical device before securing the adhesive body to the user's body.

In a ninth aspect, which may be combined with any other aspect or portion thereof described herein unless otherwise specified, the method further includes securing the adhesive body to the user's body before connecting the first strap end to the medical device; and pulling the first strap end away from the adhesive body before connecting the first strap end to the medical device.

In a tenth aspect, which may be combined with any other aspect or portion thereof described herein unless otherwise specified, the first fastener comprises a first snap button that includes a male component and a female component spaced apart on the supportive strap portion adjacent to the first strap end and are secured together to form the first fastening point, and wherein the second fastener comprises a second snap button that includes a male component and a female component spaced apart on the supportive strap portion adjacent to the second strap end and are secured together to form the second fastening point.

In an eleventh aspect, which may be combined with any other aspect or portion thereof described herein unless otherwise specified, the method further comprises threading the first strap end of the supportive strap portion through an opening of the medical device; and threading the second strap end of the supportive strap portion through an opening of the adhesive body.

In a twelfth aspect, which may be combined with any other aspect or portion thereof described herein unless otherwise specified, a male external catheter that connects to tubing of a drainage bag is provided. The male external catheter includes a tubular body, first and second supportive strap portions, and first and second adhesive bodies. The tubular body includes a first end having a first opening and a second end having a second opening, wherein the tubular member includes a flexible flange at the first end of the tubular body surrounding the first opening, the flexible flange including a first side portion and a second side portion on opposing sides of the first opening. Each first and second supportive strap portion includes a first strap end and a second strap end opposite to the first strap end. The first strap end includes a first fastener providing a first fastening point, and the second strap end includes a second fastener providing a second fastening point. The supportive strap is elastic and has an unstretched length between the first and second fastening points of the first and second strap ends. The first and second adhesive bodies, each adhesive body secured to the second strap end of the first and second supportive strap portions, respectively, using the second fasteners. The first strap ends of first and second supportive strap portions are connected to the first and second side portions, respectively, of the flexible flange using the first fasteners.

In a thirteenth aspect, which may be combined with any other aspect or portion thereof described herein unless otherwise specified, the first and second supportive strap portions are discrete components.

In a fourteenth aspect, any of the features, functionality and alternatives described in connection with any one or more of FIGS. 1 to 11 may be combined with any of the features, functionality and alternatives described in connection with any other of FIGS. 1 to 11.

Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### BRIEF DESCRIPTION OF THE DRAWINGS

The drawing figures depict one or more implementations in accord with the present concepts, by way of example only, not by way of limitations. In the figures, like reference numerals refer to the same or similar elements.

FIG. 1 is a perspective view from above of the male external catheter of the present application.

FIG. 2 is a perspective view from below of the male external catheter of FIG. 1.

FIG. 3 is a bottom plan view of the male external catheter of FIG. 1

FIG. 4 is a side elevational view of the male external catheter of FIG. 1.

FIG. 5 illustrates the male external catheter of FIG. 1 configured for a first method of use featuring a strap extending around the user's waist.

FIGS. 6A and 6B are a perspective view and a bottom plan view of the male external catheter of FIG. 1, respectively, of the present application, configured for a second method of use featuring disconnected strap ends adjacent to the catheter.

FIGS. 7A and 7B are a perspective view and a bottom plan view of the male external catheter of FIG. 1, respectively, of the present application configured for a second method of use featuring connected strap ends adjacent to the catheter.

FIGS. 8A and 8B are bottom plan and side elevational views, respectively, of the male external catheter of FIG. 1, illustrating dimensions.

FIG. 9 is an exploded view of a supportive strap portion.

FIG. 10 is a perspective view of the male external catheter of FIG. 1 of the present application configured for a third method of use featuring the supportive strap portion of FIG. 9.

FIG. 11 illustrates the male external catheter of FIG. 1 configured for the third method of use featuring the supportive strap portion of FIG. 9.

### DETAILED DESCRIPTION

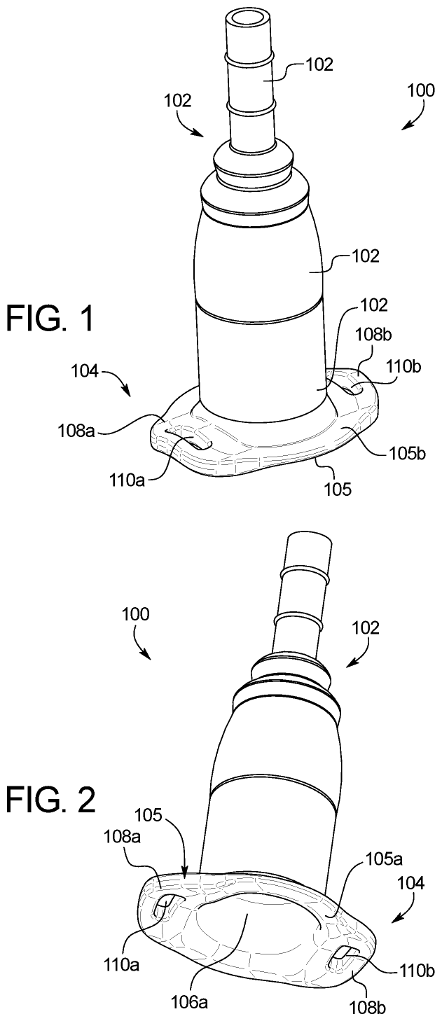

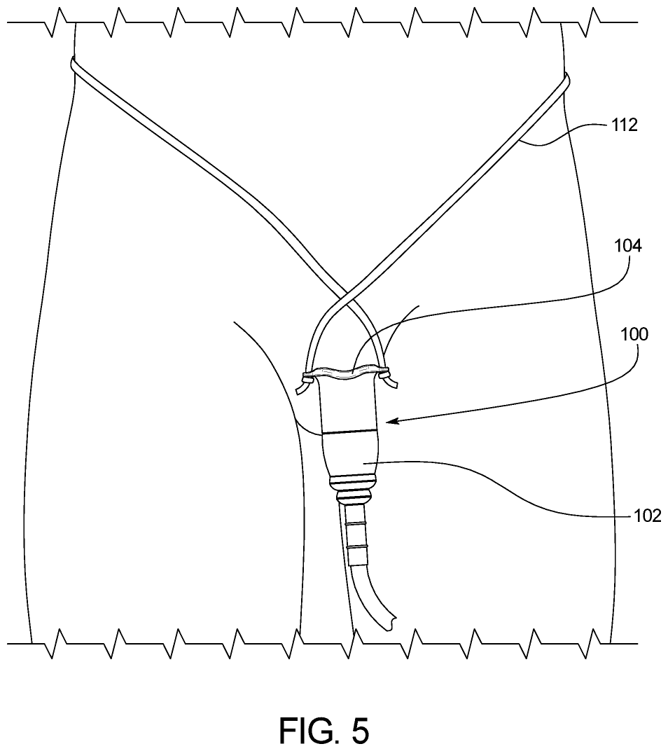

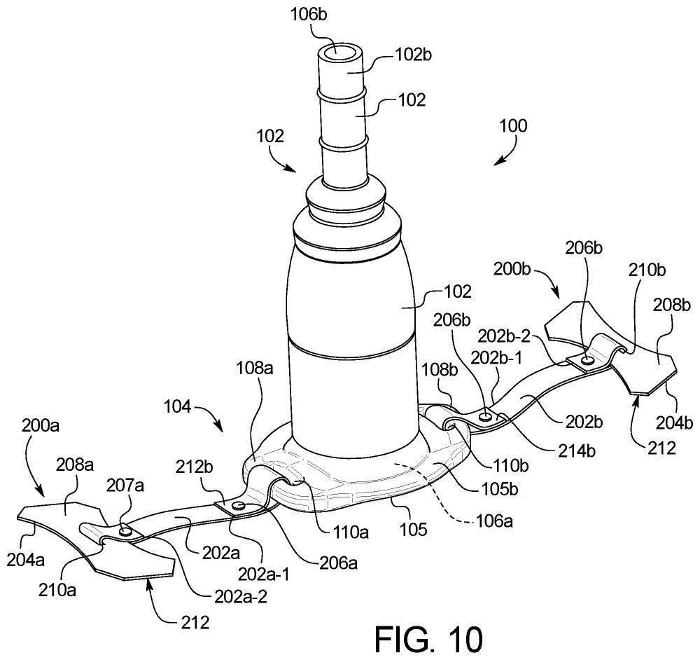

FIGS. 1-11 illustrate an example of a male external catheter 100 according to the present disclosure. Referring to FIGS. 1 and 2, the male external catheter or condom catheter 100 includes a body 102 having a tubular shape that extends from a flexible flange or base 104. The flange 104 is a body 105 providing a frame or structural support for a first opening 106a at a first end 102a of the body 102 through which the penis extends during use, as shown in FIGS. 5 and 10. The tubular shape of the body 102 of the male external catheter 100 varies in cross-section along a height H (FIG. 4) thereof. The body 102 also includes a second opening 106b at a second end 102b for connection to tubing of a drainage bag.

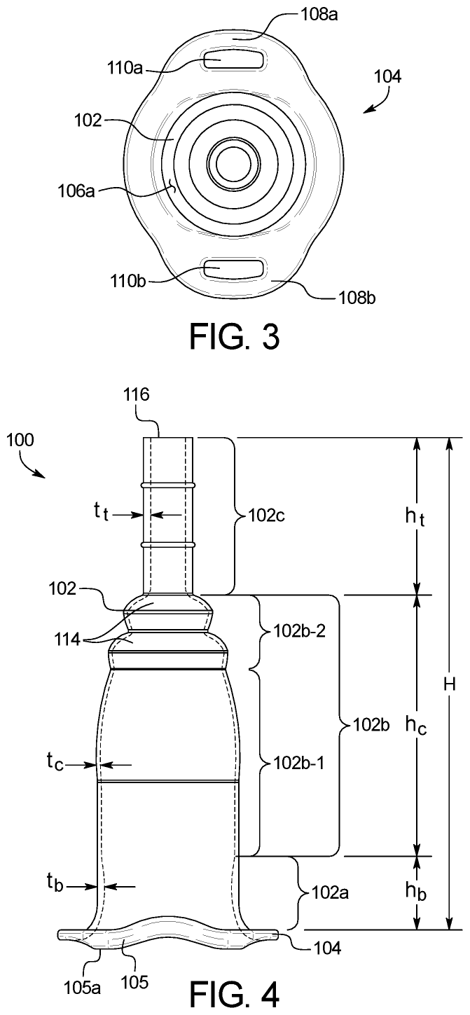

Referring to FIGS. 2 and 3, the opening 106a on the flange 104 of the male external catheter 100 defines an expandable sphincter that changes in size so that the opening 106a may conform to a diameter of a penis of the patient. The expandable sphincter provides a near liquid-tight seal between the male external catheter 100 and the penis so that urine may not easily escape. The expandable nature of the sphincter may also provide a comfortable fit for the patient and reduce the incidence of skin irritation as the body 102 of the condom catheter 100 does not slide or substantially shift when positioned on the penis.

As shown in FIG. 3, the flange 104 includes first and second side portions 108a, 108b on opposite sides of the opening 106a. Each side portion 108a, 108b has strap opening 110a, 110b for receiving a strap 112, 113 (FIGS. 5-7B). In some embodiments, the strap openings 110a, 110b have a slot shape.

During one example method of use illustrated in FIG. 5, a strap 112 may be fixedly or removably attached to the strap openings 110a, 110b of the base 104 and extend around the waist of the user to reinforce the positioning of the male external catheter 100 on the patient's body.

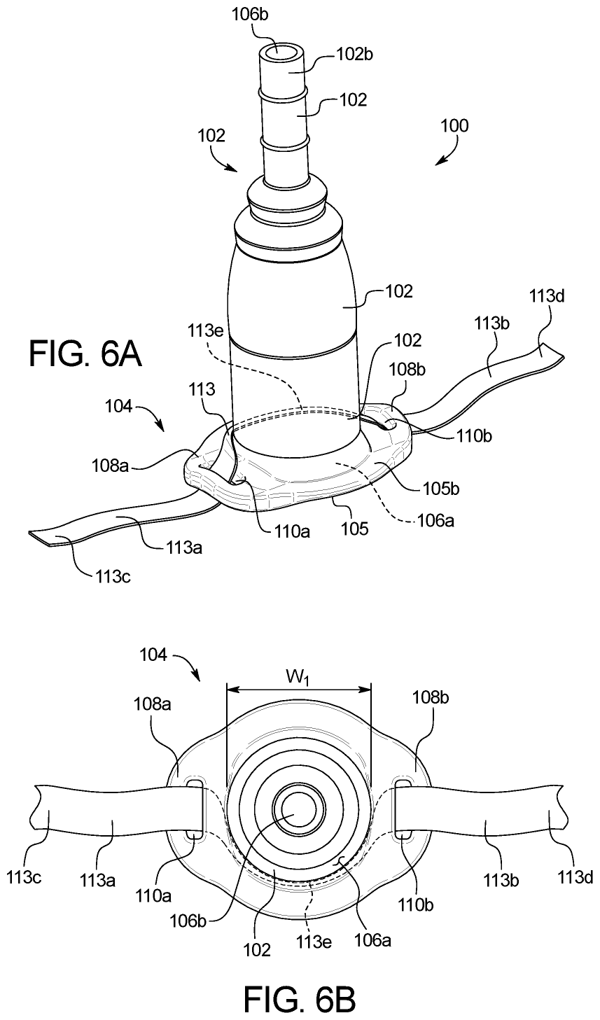

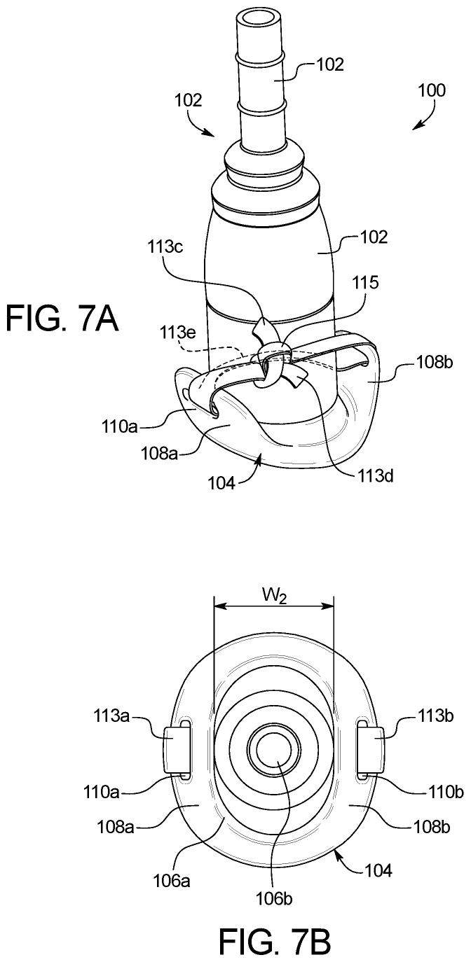

In another example method of use illustrated in FIGS. 6A-7B, a strap 113 may be fixedly or removably attached to the strap openings 110a, 110b of the flange 104 and connected together near the flange 104 so as to apply sufficient force to the first and second side portions 108a, 108b of the flange 104, respectively, to deform a shape of the opening 106a. In this embodiment, the strap 113 includes a first strap portion 113a and a second strap portion 113b connected to the first and second side portions 108a, 108b of the flange 104, respectively. The first and second strap portions 113a, 113b, respectively, include first and second strap ends 113c, 113d.

Referring to FIGS. 6A and 6B, the first and second strap ends 113c, 113d are initially disconnected such that the first and second strap portions 113a, 113b do not apply sufficient force to the first and second side portions 108a, 108b of the flange 104, respectively. The opening 106a, as a result, is not deformed and defines a first width W₁ of the opening 106a when in an undeformed configuration.

The opening 106a of the flange 104 of the male external catheter 100 is then stretched open and the penis is then inserted into the stretched, enlarged opening 106a, as described in greater detail below.

Once the male external catheter 100 is positioned on the user, the first and second strap ends 113c, 113d are connected at a point 115 as shown in FIGS. 7A and 7B such that the first and second strap portions 113a, 113b apply sufficient force to the first and second side portions 108a, 108b of the flange 104, respectively, to deform the shape of the opening 106a. Specifically, the first and second strap ends 113c, 113d connect together and cause the first and second side portions 108a, 108b of the flange 104 to bend towards the body 102 of the catheter 100, causing the opening 106a to move into a deformed configuration seen in FIG. 7B. When the strap ends 113c, 113d are connected, the deformed configuration of the opening 106a has a second width W₂ that is narrower than the first width W₁ of the undeformed configuration.

In the embodiment illustrated in FIGS. 6A-7B, the strap 113 is threaded through the strap openings 110a, 110b such that a third strap portion 113e between the first and second strap portions 113a, 113b extends below the flange 104 between the side portions 108a, 108b. During use, the third strap portion 113e extends below the flange 104 on a side opposite of the connected strap ends 113c, 113d. The third strap portion 113e provides additional reinforcement to tightly secure the opening 106a about the user's penis to prevent leaks.

When connecting the first and second strap ends 113c, 113d, the amount of tension needed is minimal in that the goal is to simply bend the side portions 108a, 108b of the flange 104 toward the body 102 of the catheter 100. Connecting the strap ends 113c, 113d too tightly and/or at a point 115 that is too close to the flange 104 may cause excessive deformation to the opening 106a and/or body 102, resulting in discomfort to the user. The amount of tension/force should be at least a minimum amount to cause the side portions 108a, 108b to bend towards the body 102 but less than a maximum amount to avoid excessive deformation and discomfort. Additionally, the first and second strap ends 113c, 113d are connected together below the flange 104 to avoid contact with the skin of the user.

After the strap ends 113c, 113d are connected, the flange 104 can be separated from the penile skin without much pressure. During use, urine flows from the body 102 through tubing into the drainage bag. A urine column forms between the body 102 of the catheter 100 and the drainage bag, and remains in place due to the negative pressure within the male external catheter 100. The user may briefly separate the flange 104 from the penile skin to allow airflow into the body 102 of the catheter 100, releasing the negative pressure therein and allowing urine within the tubing to drain into the drainage bag. The user can then allow the flange 104 to return to the deformed configuration, snugly fitting around the penis.

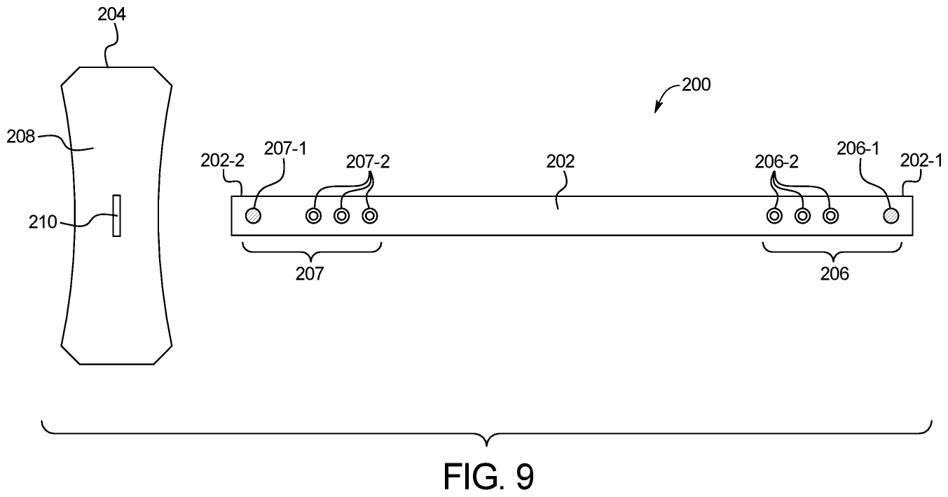

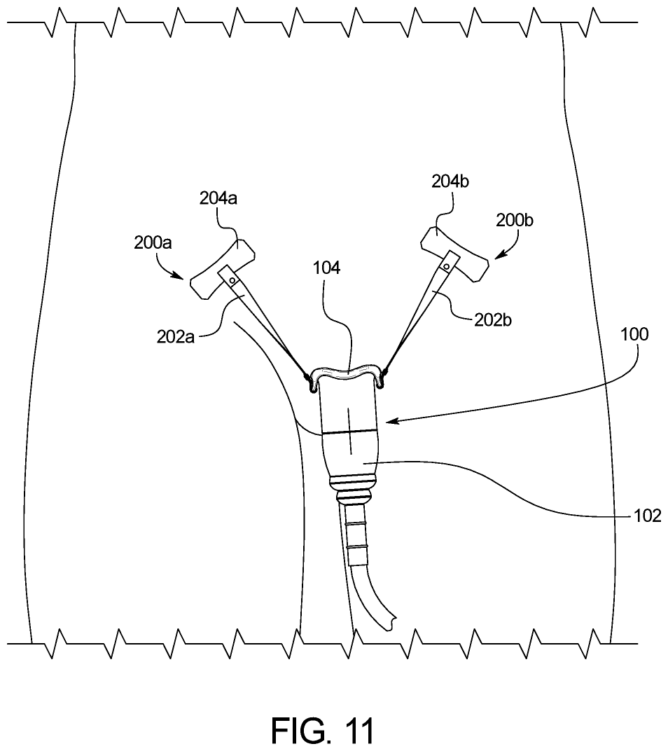

In a third method of use illustrated in FIGS. 9-11, the condom catheter 100 is further secured to the user's body using a pair of attachment devices 200. Each attachment device includes a supportive strap portion 202 with an adhesive 204 that attaches the user's body at the lower abdomen or lower anterior hip area during use as shown in FIG. 11. These attachment devices 200 maintain the positioning of the condom catheter 100 on the user's body as the user shifts and moves. In still further embodiments, the attachment device 200 may be used to secure any medical device, including but not limited to a male external catheter, a tracheostomy tube, and an endotracheal tube, to a patient.

FIG. 9 illustrates the attachment device 200 in greater detail. The supportive strap portion 202 includes a first end 202-1 and an opposite second end 202-2. In the illustrated embodiment, fasteners or snap buttons 206, 207 adjacent to the ends 202-1, 202-2 allow for easy attachment/detachment of the supportive strap portion 202 to a medical device 100 or the adhesive. This easy connection allows the user to replace the supportive strap portions 202 as needed and to separate the attachment device 200 from the medical device for washing of the medical device.

Each snap button 206, 207 includes a male component 206-1, 207-1 and at least one female component 206-2, 207-2 spaced apart by a sufficient distance, for example, approximately 4 cm, that connect together to form a fastening point. The end portion of the supportive strap portion 202 with the male component 206-1, 207-1 may fold over onto the remainder of the supportive strap portion 202 and engage with the female component 206-2, 207-2 spaced inwardly. In some embodiments, the supportive strap portion 202 includes a plurality of spaced female components 206-2, 207-2 to enable the user to adjust the length of the supportive strap portion 202 and/or the positioning of the components, such as the adhesive 204, attached to the ends 202-1, 202-2. In other embodiments, other suitable fasteners may be used. In the illustrated embodiment, the adhesive 204 includes an adhesive body 208 with an opening 210 provided therein. The opening 210 may be located centrally within the adhesive body 208 or may be positioned closer to an edge of the adhesive body 208. During use, the second end 202-2 of the supporting strap portion 202 is threaded through the opening 210 such that the male and female components 207-1, 207-2 of the snap button 207 are positioned on opposite sides of the opening 210 as shown in FIG. 9. The male and female components 206a, 206b are joined together to form a second fastening point, thereby attaching the second end 202-2 of the supportive strap portion 202 to the adhesive body 208. The opening 210 may be slotted, rounded, or have any other shape as desired.

In the illustrated embodiment, the adhesive body 208 has a butterfly shape, although suitable shape, such as rectangular, circular, etc., may be used. The size may be any size suitable for maintaining a secure attachment to the user's body, such as but not limited to 12 cm by 5 cm, 10.9 cm by 1.6 cm, 10 cm by 5 cm, 9 cm by 5 cm, or 7 cm by 2 cm.

Adhesive material 212 that adheres to the user's body is provided on an underside of the adhesive body. In some embodiments, the adhesive material 212 is a silicone adhesive for skin adhesion such as, for example, 3M™ Medical Silicone Tape 2476P, Single Sided Polyester Spunlace Nonwoven, and may retain adhesive qualities for up to five days. In some embodiments, a select area of the underside around the opening is free from adhesive material 212 and the supportive strap portion 202 contacts the underside at the select area.

In other embodiments, the adhesive body 208 may be continuous, i.e. without an opening 210. An end or other section of the supportive strap portion 202-2 may be secured directly to the underside of the adhesive body 208, and positioned between the adhesive body 208 and the user's body during use.

In a still further embodiment, the adhesive body 208 includes a loop on an outer surface of the adhesive body opposite of the underside, and the second end 202-2 of the supportive strap portion 202 is either attached to the loop or is threaded through the loop and secured thereto using a snap button or other fastener as described above.

Referring to the embodiment illustrated in FIGS. 10 and 11, the first ends 202-1 of each supportive strap portion 202 are threaded through the openings 110a, 110b of the side portions 108a, 108b of the flange 104 of the condom catheter 100. The male and female components 206-1, 206-2 of the snap buttons 206 adjacent the first ends 202-1 of the supportive strap portions 202 are positioned on opposite sides of the opening 106a and joined together to form the first fastening point, thereby securing the attachment devices 200 to the medical device 100. In some embodiments, the length of each supportive strap portion 202 is 9 in., although the length may be increased or decreased as necessary or preferred.

The supportive strap portion 202 has an unstretched overall length between the first and second strap ends 202-1, 202-2 thereof and an unstretched length between the first and second fastening points of the first and second fasteners 206, 207. When the attachment device 200 is used to attach the condom catheter 100 to the user's body as shown in FIG. 11, the adhesive body 208 is secured to the user's body while the medical device, such as the catheter 100 in FIG. 11, is simultaneously positioned on the user's body at a distance sufficiently large to cause a distance between the first and second fastening points of the first and second fasteners 206, 207 to be greater than the unstretched length between the first and second fastening points of the first and second fasteners 206, 207. This tension created along the supportive strap portion secures the medical device to the user's body.

In other embodiments, the attachment device 200 may include a relatively longer supportive strap portion 202 in lieu of two relatively shorter supportive strap portions 202. The longer supportive strap portion 202 may be threaded through the openings 110a, 110b of the side portions 108a, 108b of the flange 104 of the condom catheter 100 and extend around one side of the tubular body 102 similar to strap 113 shown in FIGS. 6A and 6B. In this embodiment, first and second adhesives 204a, 204b are attached to the first and second ends 202-1, 202-2, respectively, of the longer strap portion 202 after the longer supportive strap portion 202 is threaded through the openings 110a, 110b of the flange 104.

Referring to the embodiment illustrated in FIGS. 10 and 11, the first and second supportive strap portions 202a, 202b are discrete components. Each strap portion 202a, 202b may be formed integrally with the respective side portion 108 of the flange 104 or may be connected through a hole and peg, an adhesive, or another suitable connection means.

In the method of use of the condom catheter 100 as shown in FIGS. 9 and 10, the opening 106a of the flange remains undeformed. While some patients may prefer the first method of use illustrated in FIG. 5 or the second method of use illustrated in FIGS. 6A-7B, the method of use illustrated in FIGS. 9 and 10 eliminates the wrapping of a strap 112 around the user's body as required under the method illustrated by FIG. 5 as well as the additional pressure around the user's penis at the opening 106a in the deformed configuration as shown in FIGS. 7A and 7B. In still further embodiments, any one or more of the first, second, and third methods of use may be combined as desired and necessitated by the patient or caretaker.

In some embodiments, the straps 112, 113, 202a, 202b may comprise an elastic, non-latex, and/or silicone material. In one example, the strap 113 comprises an elastic, non-latex material having a width of about 0.5 in and a length of about 12 in. In other embodiments, the strap 112, 113, 202a, 202b may comprise an inelastic material. In still further embodiments, the strap 113 may comprise an elastic or inelastic material that connects together through a hook and loop fastener, an adhesive, or another suitable means of connection.

Referring to FIGS. 1 and 2, the body 105 includes a first surface 105a and a second surface 105b opposite the first surface 105a. In the illustrated embodiment, the first surface 105a is contoured to improve comfort and fit during use. The contoured first surface 105a is slightly concave. The body 102 of the male external catheter 100 extends from the second surface 105b, with the tubular shape thereof formed around the opening 106a of the flange 104.

Referring to FIG. 4, the body 102 includes a base portion 102a, a central portion 102b, and a tip portion 102c. The base portion 102a is adjacent to the base 104 and the tip portion 102c is distal from the base 104. The tip portion 102c is configured to connect to tubing of a drainage bag.

Each of the base portion 102a, the central portion 102b, and the tip portion 102c include a base wall thickness t_{b}, a central wall thickness t_{c}, and a tip wall thickness tₜ, respectively. In the illustrated embodiment, the central wall thickness t_{c} is less than each of the base wall thickness t_{b} and the tip wall thickness tₜ. In some embodiments, the central wall thickness t_{c} is less than one of the base wall thickness t_{b} and the tip wall thickness tₜ. This difference in wall thicknesses enables the central portion 102b of the body 102 to collapse around the user's penis while urine flows through the tip portion 102c and tip into the drainage bag.

In some embodiments, the ratio of the central wall thickness t_{c} to one or both of the base wall thickness t_{b} and the tip wall thickness tₜ is at least 1:1.1, preferably 1:1.2. In other embodiments, the ratio of the central wall thickness t_{c} to one or both of the base wall thickness t_{b} and the tip wall thickness tₜ is at least 1:1.5, and in still further embodiments, the ratio of the central wall thickness t_{c} to one or both of the base wall thickness t_{b} and the tip wall thickness tris at least 1:2.

The base portion 102a of the body 102 may be formed integrally with the base 104 and has a base height h_{b} of approximately 0.830 in, although the base height h_{b} may vary as desired or required during manufacturing. In one embodiment, the base wall thickness t_{b} is preferably about 0.060 in at the greatest point, although the base wall thickness t_{b} may vary as desired or as required during manufacturing. In one embodiment, the base wall thickness t_{b} tapers from a predominant base wall thickness t_{b-p} to the central wall thickness t_{c} near the juncture of the base portion 102a and the central portion 102b. In one embodiment, the predominant base wall thickness t_{b-p} is about 0.060 in.

The central portion 102b of the body 102 includes a planar section 102b-1 adjacent the base portion 102a and a bellowed section 102b-2 adjacent the tip portion 102c. The bellowed section 102b-2 includes horizontal bellows 114 transverse to the height H of the body 102, and the planar section 102b-1 may include optional vertical ribbing 116 connected to the horizontal bellows 114. The vertical ribs 116 may be evenly positioned along the perimeter of the body 102 near the juncture of the planar section 102b-1 and the bellowed section 102b-2.

In one embodiment, the central portion 102b has a central height h_{c} of about 1.514 in, although the central height h_{c} may vary as desired or required during manufacturing. The central wall thickness t_{c} is preferably about 0.030 in, although the central wall thickness t_{c} may vary as desired or as required during manufacturing.

The tip portion 102c of the body 102 has a tip height hₜ of about 1.315 in, although the tip height hₜ may vary as desired or required during manufacturing. The tip wall thickness tₜ is preferably about 0.060 in although the tip wall thickness tₜ may vary as desired or required during manufacturing. In some embodiments, the tip portion includes horizontal ribbing along the perimeter of the cylindrical body.

Shown in FIG. 4, the height H of the male external catheter 100 extends from the first surface 105a of the base 104 to a tip end surface 116.

The male external catheter 100 of the present application may come in different sizes to accommodate differently sized patients. While the dimensions of the structure may vary, the base wall thickness, the central wall thickness, and the tip wall thickness remain the same. In other words, while the diameter of the body may vary between sizes, the central wall thickness is preferably about 0.030 in while the base wall thickness and the tip wall thickness is about 0.060 in.

In the embodiment illustrated in FIGS. 1-10, the male external catheter 100 may have the dimensions recited in the following table in reference to FIGS. 6A and 6B for a small-medium size and a medium-large size. It is understood that the dimensions are exemplary only and do not limit the scope of any claims herein, except as may be recited thereby, together with equivalents thereof. The dimensions may vary depending during the manufacturing process or as otherwise desired.

TABLE 1

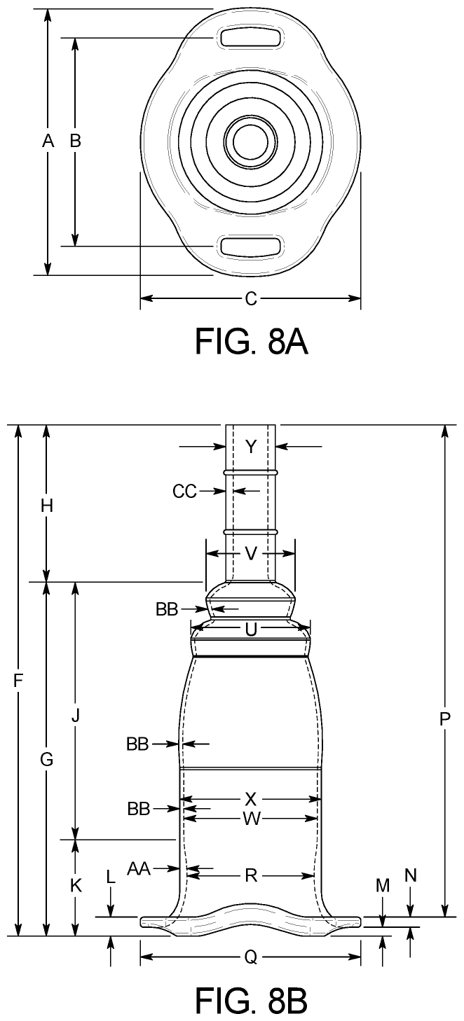

Dimensions (in)

| | Small-Medium Size | Medium-Large Size |
|---|---|---|
| FIG. 8A | | |
| A | 2.250 | 2.250 |
| B | 1.737 | 1.737 |
| C | 1.842 | 1.842 |
| FIG. 8B | | |
| F | 4.280 | 4.280 |
| G | 2.965 | 2.965 |
| H | 1.315 | 1.315 |
| J | 1.514 | 1.514 |
| K | 0.830 | 0.830 |
| L | 0.260 | 0.160 |
| M | 0.100 | 0.080 |
| N | 0.160 | 0.080 |
| P | 4.280 | 4.280 |
| Q | 1.842 | 1.607 |
| R | 0.827 | 1.062 |
| U | 1.003 | 1.003 |
| V | 0.747 | 0.747 |
| W | 0.887 | 1.122 |
| X | 0.947 | 1.182 |
| Y | 0.410 | 0.410 |
| AA | 0.060 | 0.060 |
| BB | 0.030 | 0.030 |
| CC | 0.060 | 0.060 |

In one embodiment, the male external catheter 100 is made of a silicone rubber compound, although other suitable materials may be used. The silicone rubber compound may be a peroxide-cured silicone elastomer including polydimethylsiloxane with vinyl functional groups. In one embodiment, the compound comprises approximately 70% to approximately 90% by weight polydimethylsiloxane. The silicone rubber material may be hardness (Shore A hardness test) of 20 points, a tensile strength of 800 psi, an elongation at rupture of 800%, and a specific gravity of 1.2. In some embodiments, the male external catheter 100 is latex free. In other embodiments, the body 102 and the flange 104 may be composed of the same material or different materials.

During use of the device as shown in FIG. 5, the patient is placed in a supine position and the elastic strap is positioned under the lower back of the patient. The opening 106a of the flange 104 of the male external catheter 100 is stretched open using index and middle fingers. The height H of the male external catheter 100 is compressed, using thumbs to push the tip portion 102c of the body 102 toward the base 104. The penis is then inserted into the stretched, enlarged opening 106a, and the base portion 102a of the body 102 is pulled and pushed over and above the glans penis towards the base of the penis as far as possible.

A first end of the elastic strap 112 is extended from a first side of the patient across the lower abdomen and secured to the side portion 108 opposite the first side of the patient. A second end of the elastic strap 112 is then extended from a second side of the patient across the lower abdomen and secured to the side portion 108 opposite the second side of the patient.

Referring to use of the device as shown in FIGS. 6A-7B, the patient is placed in a supine position. The opening 106a of the base 104 of the male external catheter 100 is stretched open using index and middle fingers. The height H of the male external catheter 100 is compressed, using thumbs to push the tip portion 102c of the body 102 toward the base 104. The penis is then inserted into the stretched, enlarged opening 106a, and the base portion 102a of the body 102 is pulled and pushed over and above the glans penis towards the base of the penis as far as possible.

The first end 113c and the second end 113d of the strap 113 are drawn together in front of the body 102 below the flange 104 and tied such that sufficient force is applied to the first and second side portions 108a, 108b of the flange 104, respectively, to deform a shape of the opening 106a.

In particular, the deformation of the opening 106a from the first width W₁ to the second width W₂ combined with the variation in wall thickness of the body 102 provides a male external catheter that maintains positioning on the user's penis with minimal to no leakage and without undue pressure on the penis during use. Use of the external male catheter 100 as shown in FIGS. 6A-7B eliminates the use of a strap around the body as shown in FIG. 5 as well as the use of adhesives, foam straps, or additional straps that often lead to slippage, frequency changes, and skin erosion and damage.

Further, a negative pressure exists within the body 102 of the male external catheter 100 is being used to drain urine to the drainage bag such that urine remains in the tubing between the male external catheter 100 and the drainage back. A tool, a finger, or another object may be used to separate the flange 104 from the penis of the user during use to release the negative pressure. This release of negative pressure allows the urine to flow freely through the tubing to the drainage bag, away from the male external catheter 100.

Referring to use of the device as shown in FIGS. 10 and 11, the patient is placed in a supine position. The opening 106a of the base 104 of the male external catheter 100 is stretched open using index and middle fingers. The height H of the male external catheter 100 is compressed, using thumbs to push the tip portion 102c of the body 102 toward the base 104. The penis is then inserted into the stretched, enlarged opening 106a, and the base portion 102a of the body 102 is pulled and pushed over and above the glans penis towards the base of the penis as far as possible.

The first and second strap ends 202-1, 202-2 of the supportive strap portions 202 of the attachment devices 200 are pulled upwards and away from the male external catheter 100 at an angle of about 45 degrees to position the catheter 100 onto the user's body. By using a 45 degree angle, the flange of the catheter 100 is stretched evenly on both sides thereof to keep the catheter 100 centered on the user's penis for improved functionality. For many patients that are confined to the bed, healthcare workers rotate the patient's body from side to side to prevent ulcers. Use of the supportive strap portions 202a, 202b with the adhesives 204a, 204b securely holds the catheter 100 in place on the patient during this common adjustment.

The adhesives 204a, 204b are then secured to the user's body between the lower abdomen and the lower anterior hip area, ideally in an area with minimal hair growth, in order to maintain the position of the condom catheter 100 on the body, as shown in FIG. 11. The positioning of the adhesives 204a, 204b may be adjusted as needed to accommodate the anatomy of the patient.

Conventional condom catheters typically include Velcro® (i.e., hook and loop material) and/or adhesives around the base of the penis, often constricting the penis because the materials do not expand with the changing size of the penis during use. Additionally, in some cases, use of the catheter as shown in FIGS. 6A-7B may cause discomfort in the pressure applied around the penis at the opening of the catheter 100. In other cases, use of the catheter as shown in FIG. 5 proves difficult to manage when a nurse or healthcare worker needs to physically maneuver the patient in order to wrap the strap around the patient's body, particularly when the patient is much larger than the healthcare worker. Use of the external male catheter 100 as shown in FIGS. 10 and 11 is an alternative that avoids unnecessary pressure on the user's penis and the need for wrapping the strap around the user's body, as well as avoiding the use of adhesives and/or foam straps directly on the penis or additional straps on the penis and/or body that often lead to slippage, frequency changes, and skin erosion and damage. The method of use illustrated in FIG. 11 allows for the opening in the flange of the catheter to expand and contract along with penile size during use, reducing the risk of injury.

It should be noted that various changes and modifications to the presently preferred embodiments described herein will be apparent to those skilled in the art. Such changes and modifications may be made without departing from the spirit and scope of the present invention and without diminishing its attendant advantages. For example, various embodiments of the systems and methods may be provided based on various combinations of the features and functions from the subject matter provided herein

What is claimed is:

1. An attachment device for securing a medical device to a user, the attachment device comprising:
a supportive strap portion including a first strap end and a second strap end opposite to the first strap end, wherein the first strap end includes a first fastener providing a first fastening point, wherein the second strap end includes a second fastener providing a second fastening point, and wherein the supportive strap portion is elastic and has an unstretched length between the first and second fastening points of the first and second strap ends; and
an adhesive body secured to the second strap end using the second fastener;
wherein the first strap end is connected to the medical device using the first fastener.

2. The attachment device of claim 1, wherein the first fastener comprises a first snap button that includes a male component and a female component spaced apart on the supportive strap portion adjacent to the first strap end.

3. The attachment device of claim 2, wherein the first strap end of the supportive strap portion is positioned within an opening of the medical device such that the male and female components are positioned on opposite sides of the opening of the medical device and secured together to form the first fastening point.

4. The attachment device of claim 2, wherein the second fastener comprises a second snap button that includes a male component and a female component spaced apart on the supportive strap portion adjacent to the second strap end.

5. The attachment device of claim 4, wherein each of the first and second snap buttons includes a plurality of female components positioned along the supportive strap portion.

6. The attachment device of claim 1, wherein the adhesive body includes an opening.

7. The attachment device of claim 6, wherein the second fastener comprises a second snap button that includes a male component and a female component spaced apart on the supportive strap portion adjacent to the second strap end, and the second strap end of the supportive strap portion is positioned within the opening of the adhesive body such that the male and female components are positioned on opposite sides of the opening of the adhesive body and secured together to form the second fastening point.

8. The attachment device of claim 1, further comprising:
a further supportive strap portion including a further first strap end and a further second strap end opposite to the further first strap end, wherein the further first strap end includes a first fastener providing a first fastening point, wherein the further second strap end includes a second fastener providing a second fastening point, and wherein the further supportive strap portion is elastic and has an unstretched length between the first and second fastening points of the further first and second strap ends; and
a further adhesive body secured to the further second strap end using the second fastener;
wherein the further first strap end is connected to the medical device using the first fastener.

9. The attachment device of claim 1, wherein the medical device comprises one of a male external catheter, a tracheostomy tube, and an endotracheal tube.

10. A method for securing a medical device to a user's body, the method comprising:
providing an attachment device for securing the medical device to the user's body, the attachment device comprising:
a supportive strap portion including a first strap end and a second strap end opposite to the first strap end, wherein the first strap end includes a first fastener providing a first fastening point, wherein the second strap end includes a second fastener providing a second fastening point, and wherein the supportive strap portion is elastic and has an unstretched length between the first and second fastening points of the first and second strap ends; and
an adhesive body secured to the second strap end using the second fastener;
connecting the first strap end of the supportive strap portion to the medical device using the first fastener;
positioning the medical device on the user's body; and
securing the adhesive body to the user's body;
wherein a distance between the adhesive body secured to the user's body and the medical device positioned on the user's body causes a distance between the first and second fastening points of the supportive strap portion to be greater than the unstretched length of the supportive strap portion, creating tension along the supportive strap portion that secures the medical device to the user's body.

11. The method of claim 10, further comprising:
positioning the medical device on the user's body before securing the adhesive body to the user's body; and
pulling the adhesive body and the second strap end away from the medical device before securing the adhesive body to the user's body.

12. The method of claim 10, further comprising:
securing the adhesive body to the user's body before connecting the first strap end to the medical device; and
pulling the first strap end away from the adhesive body before connecting the first strap end to the medical device.

13. The method of claim 10, wherein the first fastener comprises a first snap button that includes a male component and a female component spaced apart on the supportive strap portion adjacent to the first strap end and are secured together to form the first fastening point, and wherein the second fastener comprises a second snap button that includes a male component and a female component spaced apart on the supportive strap portion adjacent to the second strap end and are secured together to form the second fastening point.

14. The method of claim 13, further comprising:
threading the first strap end of the supportive strap portion through an opening of the medical device; and
threading the second strap end of the supportive strap portion through an opening of the adhesive body.

15. A male external catheter that connects to tubing of a drainage bag, the male external catheter comprising:
a tubular body including a first end having a first opening and a second end having a second opening, wherein the tubular member includes a flexible flange at the first end of the tubular body surrounding the first opening, the flexible flange including a first side portion and a second side portion on opposing sides of the first opening;
first and second supportive strap portion including a first strap end and a second strap end opposite to the first strap end, wherein the first strap end includes a first fastener providing a first fastening point, wherein the second strap end includes a second fastener providing a second fastening point, and wherein the supportive strap is elastic and has an unstretched length between the first and second fastening points of the first and second strap ends; and
first and second adhesive bodies, each adhesive body secured to the second strap end of the first and second supportive strap portions, respectively, using the second fasteners;
wherein the first strap ends of first and second supportive strap portions are connected to the first and second side portions, respectively, of the flexible flange using the first fasteners.

16. The male external catheter of claim 15, wherein the first and second supportive strap portions are discrete components.

17. The male external catheter of claim 15, wherein the first and second side portions of the flexible flange include first and second openings, respectively; wherein each first fastener comprises a first snap button that includes a male component and a female component spaced apart on the supportive strap portion adjacent to the first strap end; and wherein each first strap end of each supportive strap portion is positioned within the one of the first and second openings of the first and second side portions such that the male and female components of each first snap button are positioned on opposite sides of the respective opening of the flange and are secured together to form the first fastening points.
